(19) United States
(12) Patent Application Publication
(10) Pub. No.: US 2009/0325252 A1
(43) Pub. Date: Dec. 31, 2009

(54) Process of treating cellulosic biomass material to produce ethanol

(76) Inventor: Donald L. Law, Post Falls, ID (US)

Correspondence Address: DONALD L. LAW, 2811 E Ferry Landing, Post Falls, ID, 83854, US

(21) Appl. No.: 12/163,705

(22) Filed: Jun. 27, 2008

(51) Int. Cl. C12P 7/14

(52) U.S. Cl. 435/162

(57) ABSTRACT

This invention relates to a process of treating a cellulosic biomass material to produce ethanol, at atmospheric temperatures and pressures. This process does not utilize high pressures or temperatures. The process includes the steps of: (a) exposing the cellulosic biomass material to acetic acid [C₂H₄O₂] for 1-2 days, to produce a step (a) product, (b) exposing the step (a) product to a second treatment consisting of orthophosphoric acid [H₃PO₄] and hydrogen peroxide [H₂O₂], at atmospheric pressure to produce a step (b) product; and (c) further processing the step (b) product to produce ethanol and other co-products.

### BACKGROUND OF THE INVENTION

This invention relates in general to processes for producing ethanol from a cellulosic biomass material.

Fossil fuels are the dominant source of energy for industry and consumers. The result has been a contaminated environment, dependence on unstable nations for energy supplies and the volatile nature of prices as fossil fuel sources become scarce.

Ethanol, is a naturally renewable fuel source, that has the potential to replace a high percentage of fossil fuels being imported. The move to ethanol-enriched cleaner fuels, which eliminate or reduce the polluting and carcinogenic additives required to enhance gasoline, has produced a huge and increasing demand for ethanol around the world.

With the increasing cost of oil and gasoline and the limitations of grain-ethanol which is competing with food resources, cellulosic ethanol has become commercially viable. There is tremendous growth forecasted in this area and the Renewable Fuel Standard Energy Policy Act of 2005, has mandated the production of ethanol to 36 Billion gal/yr by 2022, providing tax incentives.

Presently, almost all ethanol production facilities in North America are corn- or grain-based. They grind up starch/carbohydrate rich corn/grain, treat this with a complex process to break raw biomass cellulose, and then ferment the sugars into ethanol (with the by-product of CO₂) for industrial/commercial and medical uses.

The technology advances in the grain-ethanol production have reduced the, cost of ethanol to competitive levels, but they are reaching a production limit which is related to the availability and price of feedstock, such as corn. A major expense and major factor will always be the price of corn/grain (corn recently reaching a price of $4.00/bushel). It has been determined by DOE, that the supply is far too limited to allow for the competing worldwide demand for both food and ethanol in the future.

In view of these facts, nations such as USA, Canada and China have in the last two years initiated nationwide efforts to investigate and develop technology to produce ethanol. from lignocellulosic biomass (e.g., saw dust, waste paper, wood chips, leaves, corn stover, straw, bagasse, rice straw, and municipal cellulosic waste). Canada is constructing a cellulosic ethanol demonstration plant in Ottawa and the Canadian company is negotiating a joint-venture for another plant in China. In a typical lignocellulosic biomass process, the feedstock primarily composed of cellulose is ground up and then pre-treated (usually with acid) to break down the cellulose and separate the three main components of wood (cellulose, hemi-cellulose and lignin). These components are then followed by hydrolysis and fermentation. The application of cellulases to hydrolyze plant-cell polysaccharides, produces a mixture of simple sugars. The fermentation is mediated by bacteria or yeast, to convert the sugars into ethanol and other co-products. The main advantage of this process is that there is an unlimited supply of lignocellulosic biomass of many types, it is fully renewable and natural, and it is inexpensive. In fact, many potential sources of lignocelluosic biomass actually generate revenue for the process due to their present disposal costs. Bio-ethanol production is relatively environmental friendly, as much of this feedstock material is burned, ploughed under or composted. However, based on present technologies, the current cost/gallon for bio-ethanol remains high in relation to fossil fuels. Lignocellulosic ethanol production until recently was costly, due to the fact that cellulosic feedstocks were difficult and expensive to break down into fermentable materials. With the advancement of R&D activities by the US Department of Energy (DOE), The Office of Energy Efficiency and Renewable Energy, in the last ten years, new thermochemical technologies for solubilizing hemicellulosic sugars have been developed making cellulosic sugars more accessible to hydrolysis enzymes and fermentation organisms.

There is an extensive patent literature relating to de-lignification of lignocellulosic materials, predominantly relating to applications in the pulp and paper industry. For example, bleaching of lignocellulosic materials in the presence of oxygen and peroxide has been described in U.S. patents such as Gould U.S. Pat. No. 4,649,113, Holtzapple U.S. Pat. No. 5,865,898, Farley U.S. Pat. No. 3,719,552, Tyson U.S. Pat. No. 4,842,877, Phillips U.S. Pat. No. 4,372,812, Paren U.S. Pat. No. 6,165,318, Francis U.S. Pat. No. 4,729,817, Miller U.S. Pat. No. 5,916,415, Foody U.S. Pat. No. 6,090,595, and in Patent pending such as Forslund 20010025695.

The described processes focus on improvement in de-lignification during bleaching of paper pulps with retention of viscosity index (indicative of cellulose strand integrity/predictive of paper strength). The primary goal of these de-lignification process improvements in the pulp and paper industry is to de-lignify with reduced disruption of the cellulose polymer structure. Most of this work is not related to pretreatment during ethanol production.

### SUMMARY OF THE INVENTION

This invention relates to a process of treating a lignocellulosic biomass material to produce ethanol. The process includes the steps of: (a) exposing the lignocellulosic material to acetic acid for 1-2 days, to produce a step (a) product, (b) exposing the step (a) product to a second treatment consisting of phosphoric acid and hydrogen peroxide, at atmospheric pressure and temperature to produce a step (b) product; and (c) further processing the step (b) product to produce ethanol.

Various advantages of this invention will become apparent from the following detailed description of the preferred embodiments.

### BRIEF DESCRIPTION OF THE DRAWING

FIG. 1 is a flow diagram of a preferred process of treating a lignocellulosic biomass material to produce ethanol in accordance with the present invention.

### DETAILED DESCRIPTION OF THE PREFERRED EMBODIMENTS

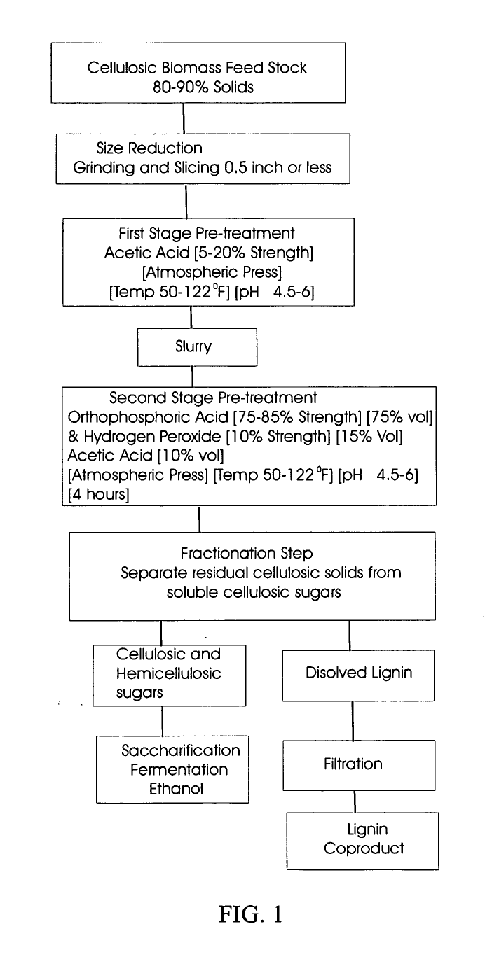

This invention relates to an improved process of treating a lignocellulosic biomass material to produce ethanol. Any suitable lignocellulosic material can be used in the invention. As shown in FIG. 1, a preferred lignocellulosic feed stock contains from about 80%-90% solids by weight. Preferably, the lignocellulosic material is lignocellulosic biomass, such as paper waste, wood chips, sawdust, leaves, corn stover, straw, bagasse, rice straw, municipal solid cellulosic waste, and/or other known lignocellulosic biomass.

As shown in FIG. 1, preferably the lignocellulosic material is prepared for the size reduction process by grinding and/or slicing. Any suitable grinding or slicing equipment and process can be used.

The lignocellulosic material is then exposed to acetic acid for 1-2 days. The acid treatment helps to break down the lignocellulosic material. Preferably, the acetic acid should have a 5%-20% strength just above the biomass material.

A secondary treatment of the slurry produced consists of exposing the slurry to a 75%-85% strength of orthophosphoric acid [H₃PO₄] at 75% volume and a 10% strength hydrogen peroxide [H₂O₂] at 15% volume and Acetic acid [C₂H₄O₂] in 10% volume in the slurry. This mixture is then exposed to atmospheric temperatures and pressures for a time lapse of 4 hours. The resulting product will undergo a fractionation step, being screened and filtered by a by a system such as screen & screw dewaterer, the solid products will be transferred to a solid pile and the juice to a fermentation tank where the pH will be maintained between 4.5 and 6.

The pH can be achieved by the addition of any suitable alkaline material, such as KOH or CaCO₃. Preferably, the conditions also include a temperature not lower than about 50° F. The focus of this treatment is on optimal de-lignification of the material and the provision of the maximum number of cellulase binding sites (theoretically reducing the amount and increasing the efficiency of enzyme action on the substrate).

The product usually consists of a slurry containing cellulose polymers, solubulized lignin, hemicellulose polymerssuch as pentose compounds and other materials.

The flow diagram in FIG. 1 shows some of these preferred process conditions in the boxes entitled “First Stage Pre-Treatment” and “Second Stage Pre-treatment”. The treatment conditions would be detrimental to the production of paper where integrity of the long cellulose strands and limited saccharification is beneficial. In contrast to the process conditions used for pulp and paper manufacturing, the process conditions of the present invention are aimed at disconstructing the lignocellulosic structure and breaking up the long cellulose polymers, make them more accessible to enzymatic breakdown and free up hemicellulosic sugars.

The slurry from the above process is subjected to fermentation mediated by bacteria or yeast, to convert sugars to ethanol.

As shown in FIG. 1, the filtered cellulosic solids are suitable for efficient fermentation and saccharification.

The alkaline liquid stream should be easily separated by raising the pH to force precipitation of the lignin which can then be filtered, and either fermentation or further separation of the water soluble sugars for ethanol.

In accordance with the provisions of the patent statutes, the principle and mode of operation of this invention have been explained and illustrated in its preferred embodiment. However, it must be understood that this invention may be practiced otherwise than as specifically explained and illustrated without departing from its spirit or scope.

What is claimed is:

1. A process of treating lignocellulosic biomass material to produce ethanol, the process comprising the steps of: (a) size reduction by grinding and/or slicing to particle size 0.5 inch or less, a quantity of untreated cellulosic biomass material; (b) exposing the reduced fibers of untreated lignocellulosic material of step (a) to conditions including an acetic acid [C₂H₄O₂] treatment at atmospheric pressure to produce a step (b) product; (c) exposing the (b) product to a second treatment of orthophosphoric acid [H₃PO₄] and hydrogen peroxide [H₂O₂], at ambient temperature, to produce a slurry containing solids; and (d) further processing said slurry by passing said slurry through a device to separate the cellulosic solids from a liquid stream and (e) further processing the step (d) liquid product to produce ethanol.

2. A process according to claim 1 wherein the conditions of step (c) include an ambient temperature between 50° F.-122° F.

3. A process according to claim 1, wherein the conditions of step (b) include acetic acid.

4. A process according to claim 1, wherein the conditions of step (c) include orthophosphoric acid and hydrogen peroxide.

5. A process according to claim 1, wherein step (b) is conducted from a time of from about 1 hour to 2 days.

6. A process according to claim 1, wherein step (c) is conducted from a time of from about 4 hours depending on the biomass type.

7. A process according to claim 1, wherein the further processing of step (d) includes saccharification, fermentation using the white rot fungus “trametes versicolor (L.Fr.) pilat” and Saccharomyces exiguus.
